(19) United States
(12) Patent Application Publication
(10) Pub. No.: US 2017/0304097 A1
(43) Pub. Date: Oct. 26, 2017

(54) STENT-GRAFT DELIVERY SYSTEM HAVING AN INNER SHAFT COMPONENT WITH A LOADING PAD OR COVERING ON A DISTAL SEGMENT THEREOF FOR STENT RETENTION

(71) Applicant: Medtronic Vascular, Inc., Santa Rosa, CA (US)

(72) Inventors: Lisa Corwin, Santa Rosa, CA (US); Peter Larson, Minneapolis, MN (US); Robert Murray, III, Santa Rosa, CA (US); Francesco Piccagli, Roncadelle (IT); Theresa Wittrock, Galway (IE)

(21) Appl. No.: 15/134,427

(22) Filed: Apr. 21, 2016

(51) Int. Cl. A61F 2/966; A61B 17/00; A61F 2/90; A61F 2/07

(52) U.S. Cl. CPC A61F 2/966; A61F 2/90; A61F 2/07; A61B 17/00234; A61B 2017/00292; A61B 2017/00862

(57) ABSTRACT

A catheter for delivering a self-expanding stent-graft prosthesis includes an inner shaft component having a distal segment over which the stent-graft prosthesis is loaded in a compressed delivery configuration. The distal segment has a covering that is intimately disposed thereover. The covering forms a raised outer surface on the distal segment and includes a plurality of protrusions that extend radially outward from the raised outer surface. An outer shaft component is slidingly disposed over the distal segment of the inner shaft component for holding the stent-graft prosthesis in the compressed delivery configuration. In the compressed delivery configuration, the stent-graft prosthesis is disposed over and makes contact with the covering of the distal segment of the inner shaft component such that at least the plurality of protrusions provide sufficient friction to of the covering secure a longitudinal position of the stent-graft prosthesis relative to the inner shaft component.

### FIELD OF THE INVENTION

The invention is related in general to implantable prostheses and in particular to self-expanding stent-grafts.

### BACKGROUND OF THE INVENTION

Prostheses for implantation in blood vessels or other similar organs of the living body are, in general, well known in the medical art. For example, prosthetic endovascular grafts constructed of biocompatible materials have been employed to replace or bypass damaged or occluded natural blood vessels. In general, endovascular grafts include a graft anchoring component that operates to hold a tubular graft component of a suitable graft material in its intended position within the blood vessel. Most commonly, the graft anchoring component is one or more radially compressible stents that are radially expanded in situ to anchor the tubular graft component to the wall of a blood vessel or anatomical conduit. Thus, endovascular grafts are typically held in place by mechanical engagement and friction due to the opposition forces provided by the radially expanded stents.

Grafting procedures are also known for treating aneurysms. Aneurysms result from weak, thinned blood vessel walls that “balloon” or expand due to aging, disease and/or blood pressure in the vessel. Consequently, aneurysmal vessels have a potential to rupture, causing internal bleeding and potentially life threatening conditions. Grafts are often used to isolate aneurysms or other blood vessel abnormalities from normal blood pressure, reducing pressure on the weakened vessel wall and reducing the chance of vessel rupture. As such, a tubular endovascular graft may be placed within the aneurysmal blood vessel to create a new flow path and an artificial flow conduit through the aneurysm, thereby reducing if not nearly eliminating the exertion of blood pressure on the aneurysm.

In general, rather than performing an open surgical procedure to implant a bypass graft that may be traumatic and invasive, endovascular grafts which may be referred to as stent-grafts are preferably deployed through a less invasive intraluminal delivery procedure. More particularly, a lumen or vasculature is accessed percutaneously at a convenient and less traumatic entry point, and the stent-graft is routed through the vasculature to the site where the prosthesis is to be deployed. Intraluminal deployment is typically effected using a delivery catheter with coaxial inner and outer tubes or shafts arranged for relative axial movement. For example, a self-expanding stent-graft may be compressed and disposed within a distal end of an outer shaft or sheath component of the delivery catheter. The delivery catheter is then maneuvered, typically tracked through a body lumen until a distal end of the delivery catheter and the stent-graft are positioned at the intended treatment site. The inner tube is then held stationary while the sheath of the delivery catheter is withdrawn. As the sheath component is withdrawn, the stent-graft is released from the confines thereof and radially self-expands so that at least a portion of it contacts and substantially conforms to a portion of the surrounding interior of the lumen, e.g., the blood vessel wall or anatomical conduit.

In some applications, self-expanding stent-grafts lack axial strength and as a result, undesirable longitudinal or axial movement such as but not limited to shifting, wrinkling, bunching-up or elongation of the stent-graft may occur during loading and/or deployment thereof. Embodiments hereof are directed to a delivery system for self-expanding stent-grafts that prevents or minimizes the above-mentioned undesirable longitudinal or axial movement of the stent-graft.

### BRIEF SUMMARY OF THE INVENTION

Embodiments hereof relate to a catheter for delivering a self-expanding stent-graft prosthesis. The catheter includes an inner shaft component having a distal segment over which the stent-graft prosthesis is loaded in a compressed delivery configuration. The distal segment has a covering that is intimately disposed thereover. The covering forms a raised outer surface on the distal segment and includes at least one protrusion that extends radially outward from the raised outer surface. The catheter further includes an outer shaft component slidingly disposed over the distal segment of the inner shaft component for holding the stent-graft prosthesis in the compressed delivery configuration. In the compressed delivery configuration, the stent-graft prosthesis is disposed over and makes contact with the covering of the distal segment of the inner shaft component such that at least the at least one protrusion provides sufficient friction to the covering to secure a longitudinal position of the stent-graft prosthesis relative to the inner shaft component.

Embodiments hereof also relate to a stent-graft delivery system including an inner shaft component, an outer shaft component, and a self-expanding stent-graft prosthesis. The inner shaft component has a proximal segment and a distal segment. The distal segment has a plurality of protrusions that radially extend away from the outer surface of the inner shaft component. The self-expanding stent-graft prosthesis is disposed over the distal segment. The self-expanding stent-graft prosthesis has a radially compressed configuration for delivery within a vasculature and a radially expanded configuration for deployment within a body lumen. The outer shaft component is slidingly disposed over the distal segment of the inner shaft component for holding the self-expanding stent-graft prosthesis in the radially compressed configuration. The plurality of protrusions secure a longitudinal position of the self-expanding stent-graft prosthesis relative to the inner shaft component during loading and deployment of the self-expanding stent-graft prosthesis.

Embodiments hereof also relate to a method of loading a self-expanding stent-graft prosthesis into a delivery catheter. A self-expanding stent-graft prosthesis is radially compressed onto a loading pad on a distal segment of an inner shaft component of the delivery catheter. The loading pad is intimately disposed over the distal segment and defines a raised outer surface of the distal segment and a plurality of protrusions that extend radially away from the raised outer surface. The inner shaft component with the self-expanding stent-graft prosthesis radially compressed thereon is positioned within a lumen of an outer shaft component. At least the plurality of protrusions secure a longitudinal position of the self-expanding stent-graft prosthesis relative to the inner shaft component such that the self-expanding stent-graft prosthesis does not move relative to the inner shaft component while the inner shaft component is being positioned within the lumen of the outer shaft component.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a stent-graft delivery system according to an embodiment hereof, wherein an outer shaft component of the stent-graft delivery system is disposed over a stent-graft prosthesis in a compressed delivery configuration, the stent-graft prosthesis being disposed over a covering or loading pad that is intimately disposed over a distal segment of an inner shaft component of the stent-graft delivery system and wherein protrusions of the covering or loading pad are bumps and a distal portion of the stent-graft delivery system is shown in section.

FIG. 1A is a cross-sectional view of the stent-graft delivery system of FIG. 1 taken along line A-A of FIG. 1.

FIG. 2 is an enlarged sectional view of a portion of the distal segment of the inner shaft component of the stent-graft delivery system of FIG. 1, wherein the stent-graft prosthesis is not shown in the sectional view for illustrative purposes only.

FIG. 3 is a side view of the inner shaft component of the stent-graft delivery system of FIG. 1, wherein the inner shaft component is removed from the stent-graft delivery system for illustrative purposes only.

FIG. 4 is a side view of a distal segment of an inner shaft component of a stent-graft delivery system according to another embodiment hereof, wherein protrusions of a covering or loading pad that is intimately disposed over the distal segment are circumferential bands.

FIG. 4A is an enlarged side view of a portion of a circumferential band of the covering of FIG. 4.

FIG. 4B is a side view of a distal segment of an inner shaft component of a stent-graft delivery system according to another embodiment hereof, wherein a single protrusion of a covering or loading pad is intimately disposed over the distal segment of the inner shaft component.

FIG. 5 is a side view of a distal segment of an inner shaft component of a stent-graft delivery system according to another embodiment hereof, wherein a plurality of protrusions are intimately and directly disposed over the inner shaft component.

FIG. 6 is a perspective view of an exemplary stent-graft prosthesis that may be used with the stent-graft delivery system of FIG. 1, wherein the stent-graft prosthesis is in a deployed or radially expanded configuration.

FIG. 7 is a perspective view of another exemplary stent-graft prosthesis that may be used with the stent-graft delivery system of FIG. 1, wherein the stent-graft prosthesis is in a deployed or radially expanded configuration.

### DETAILED DESCRIPTION OF THE INVENTION

Specific embodiments are now described with reference to the figures, wherein like reference numbers indicate identical or functionally similar elements. Unless otherwise indicated, for the delivery system the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” is used in the following description with reference to one or more stents or scaffolds of the prostheses hereof and is intended to convey that the structures are shaped or formed from a material that can be provided with a mechanical memory to return the structure from a compressed or constricted delivery configuration to an expanded deployed configuration. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in embodiments hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

Embodiments hereof relate to a catheter for delivering a self-expanding stent-graft prosthesis. The catheter includes an inner shaft component having a distal segment which has a covering or loading pad that is intimately disposed thereover. The covering forms a raised or built-up outer surface on the distal segment and includes a plurality of protrusions that extend radially outward from the raised outer surface. The raised outer surface and the plurality of protrusions secure a longitudinal position of the self-expanding stent-graft prosthesis relative to the inner shaft component during loading and deployment of the self-expanding stent-graft prosthesis. Rather than a stent stop as known in the prior art, embodiments hereof utilizes the covering to lock or secure the stent-graft prosthesis in place during loading and deployment thereof.

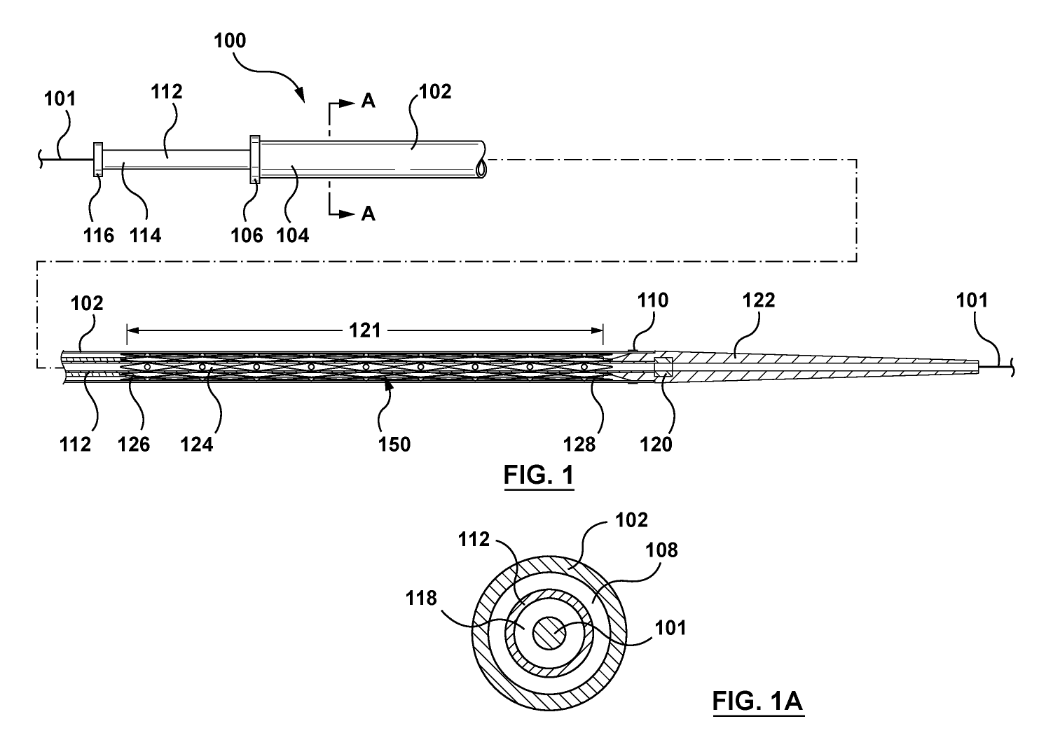

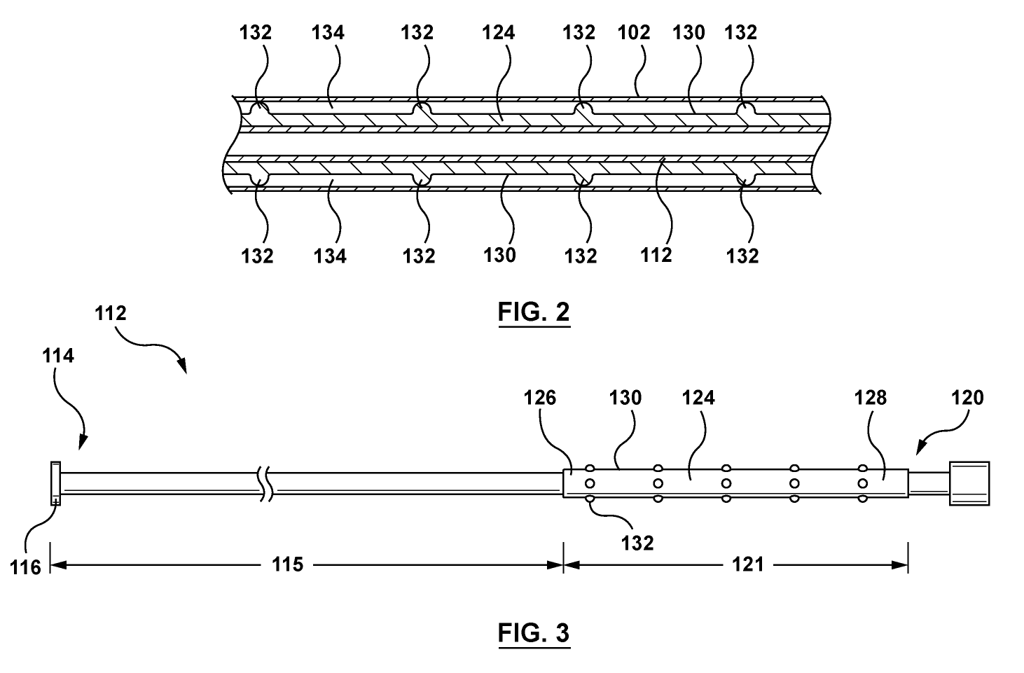

More particularly, a stent-graft delivery system 100 according to an embodiment hereof will be described with reference to FIGS. 1, 1A, 2, and 3. FIG. 1 is a side view of stent-graft delivery system 100 with a distal portion thereof shown in section, FIG. 1A is a cross-sectional view of stent-graft delivery system 100 taken along line A-A of FIG. 1, FIG. 2 is an enlarged sectional view of a portion of stent-graft delivery system 100, and FIG. 3 is a side view of inner shaft component 112 of stent-graft delivery system 100, the inner shaft component being removed from the stent-graft delivery system for illustrative purposes only.

Stent-graft delivery system 100 includes an elongate inner shaft component 112 having a handle 116 coupled to a proximal end 114 thereof and a distal tip component 122 coupled to a distal end 120 thereof, and an outer shaft component or sheath 102 sliding disposed over inner shaft component 112 to retain a self-expanding stent-graft prosthesis 150 in a radially constrained or compressed delivery configuration while the delivery system is tracked through a body lumen to the deployment site. In FIG. 1, outer shaft component 102 is in a non-retracted, delivery configuration and extends over stent-graft prosthesis 150 to restrain the stent-graft prosthesis in the compressed delivery configuration. In FIG. 2, stent-graft prosthesis 150 is not shown for illustrative purposes only. Outer shaft component 102 defines a lumen 108 extending from a proximal end 104 to a distal end 110. Outer shaft component 102 is movable in an axial direction along and relative to inner shaft component 112 and extends to a proximal portion of the stent-graft delivery system where it may be controlled via an actuator, such as a handle 106 to selectively expand stent-graft prosthesis 150 disposed around inner shaft component 112. Handle 106 may be a push-pull actuator that is attached or connected to a proximal end 104 of outer shaft component 102. Alternatively, the actuator may be a rotatable knob (not shown) that is attached or connected to proximal end 104 of outer shaft component 102 such that when the knob is rotated, outer shaft component 102 is retracted in a proximal direction to expand the graft. Alternatively, the actuator may use a combination of rotation and sliding to retract outer shaft component 102, as described, for example, in U.S. Pat. No. 7,419,501 to Shiu et al., U.S. Patent Publication No. 2011/0257718 to Argentine, U.S. Patent Publication No. 2011/0270371 to Argentine, and U.S. Patent Publication No. 2011/0270372 to Argentine, each of which is incorporated by reference herein. Thus, when the actuator is operated, i.e., manually turned or pulled, outer shaft component 102 is proximally retracted over inner shaft component 112 in a proximal direction. Outer shaft component 102 may be constructed of any suitable flexible polymeric material, including but not limited to polyethylene terephalate (PET), nylon, polyethylene, PEBAX, or combinations thereof, either blended or co-extruded.

Inner shaft component 112 may be constructed from a rigid plastic tube of PEEK polyetheretherketone, polyimide, a Nickel-Titanium alloy, or similar polymeric or metallic materials. Inner shaft component 112 may define a guidewire lumen 118 for receiving a guidewire 101 there through. Alternatively, inner shaft component 112 may instead be a solid rod (not shown) without a lumen extending there through. In an embodiment where inner shaft component 112 is a solid rod, inner shaft component 112 is tracked to the target site with the assistance of a tapered and flexible nosecone of distal tip component 122. Suitable materials for distal tip component 122 include PEBAX, urethane, silicone, other flexible polymers, and the like, any of which may also include a radiopaque additive to provide the clinician with a visible tip when using fluoroscopy guidance to deliver the stent-graft within the patient.

With additional reference to FIG. 3, inner shaft component 112 includes an elongate proximal segment 115 and a distal segment 121. A covering or loading pad 124 is intimately disposed over distal segment 115 of inner shaft component 112. As used herein, “intimately disposed” means that covering 124 is fixedly or integrally disposed or formed over inner shaft component 112 such that the covering 124 is permanently and non-removably attached to the inner shaft component. Covering 124 extends around or covers the entire circumference of inner shaft component 112. In an embodiment hereof, covering 124 is an outer layer, jacket, or coating formed on distal segment 115 of inner shaft component 112 such that the covering forms or defines a raised outer surface 130 relative to proximal segment 115 of inner shaft component 112. In an embodiment, inner shaft component 112 is formed from a tubing of a first material and covering 124 is formed from a second material different from the first material. More particularly, inner shaft component 112 may be formed from a tubing of a non-elastomeric material and covering 124 is formed from an elastomeric material. Suitable materials for covering 124 include PEBAX, nylon, acrylonitrile butadiene styrene (ABS), polycarbonate, silicones, urethanes, polymeric rubbers, and similar materials.

Covering 124 further includes a plurality of protrusions 132 that extend radially outward from raised outer surface 130. In the embodiment of FIGS. 1-3, each protrusion 132 is a bump having a generally cylindrical, semi-spherical, dome-shaped, or conical configuration. The plurality of protrusions 132 may be regularly or equally spaced over a length of raised outer surface 130. Stated another way, covering 124 has a first or proximal end 126 and a second or distal end 128 and the plurality of protrusions 132 may be regularly or equally spaced between first and second ends 126, 128 of the covering. Those skilled in the art will appreciate that covering 124 and protrusions 132 can be formed as a single unit and/or assembled from individual parts or components. Stated another way, the plurality of protrusions 132 may be integrally formed with covering 124 or may be separately formed from covering 124 and subsequently attached to covering 124. In an embodiment hereof, an elastomeric layer of material is intimately disposed over distal segment 115 of inner shaft component 112 to form covering 124 and the plurality of protrusions 132.

In the compressed delivery configuration, stent-graft prosthesis 150 is disposed over and makes contact with covering 124 such that at least the plurality of protrusions 132 provide sufficient friction to covering 124 to secure a longitudinal position of stent-graft prosthesis 150 relative to inner shaft component 112. In addition, covering 124 itself which is preferably formed from an elastomeric material also provides friction to secure a longitudinal position of stent-graft prosthesis 150 relative to inner shaft component 112. Covering 124 modifies and builds up inner shaft component 112 to enhance the interface and adhesion between stent-graft prosthesis 150 and stent-graft delivery system 100 during loading and deployment of stent-graft prosthesis 150. Covering 124 passively grips stent-graft prosthesis 150 by increasing the surface friction between stent-graft prosthesis 150 and distal segment 121 of inner shaft component 112. Further, the plurality of protrusions 132 provide a stop or frictional force for the crowns of stent-graft prosthesis 150 during loading and/or deployment of outer shaft component 102. When disposed over inner shaft component 112, the crowns of stent-graft prosthesis 150 are positioned in spaces or gaps 134 (best shown on FIG. 2 when stent-graft prosthesis 150 is not shown for sake of illustrating gaps 134) between protrusions 132 and thus movement of the crowns of stent-graft prosthesis 150 is restricted by the plurality of protrusions 132. Protrusions 132 also provide friction to/act as opposition to the graft material of stent-graft prosthesis 150.

More particularly, covering 124 and the plurality of protrusions 132 prevent movement of stent-graft prosthesis 150 relative to inner shaft component 112 during loading of stent-graft prosthesis 150 into outer shaft component 102 for delivery thereof. Some stent designs have a tendency to bunch or compress axially during loading. Stent-graft prosthesis 150 is compressed or crimped onto distal segment 121 of inner shaft component 112 having covering 124 and the plurality of protrusions 132. Once compressed or crimped onto covering 124 and the plurality of protrusions 132, the longitudinal or axial position of stent-graft prosthesis 150 is set, locked, secured, or certain relative to inner shaft component 112 when inner shaft component 112 with compressed stent-graft prosthesis 150 mounted thereon is pulled within outer shaft component 102 during loading. As such, the compressed self-expanding stent-graft prosthesis does not move, shift, wrinkle, bunch-up or elongate anywhere along its length but instead stays in its original longitudinal or axial position during loading.

Covering 124 and the plurality of protrusions 132 also prevent movement of stent-graft prosthesis 150 relative to inner shaft component 112 during deployment of stent-graft prosthesis 150 within the body lumen when outer shaft component 102 is proximally retracted relative to inner shaft component 112 to release stent-graft prosthesis 150. When outer shaft component 102 is proximally retracted to expose stent-graft prosthesis 150, stent-graft prosthesis 150 deploys radially outward at a certain deployed length. The increased friction due to covering 124 and the plurality of protrusions 132 holds stent-graft prosthesis 150 in place within delivery system 100 during retraction of outer shaft component 102 (for deployment of stent-graft prosthesis 150) to enhance the control and accuracy of deployment of stent-graft prosthesis 150. More particularly, some stent designs have a tendency to bunch or compress axially during deployment. The increased friction between stent-graft prosthesis 150 and inner shaft component 112 due to covering 124 helps to maintain the length and position of stent-graft prosthesis 150 as outer shaft component 102 is retracted. Thus, covering 124 prevents stent-graft prosthesis 150 from slipping or ejecting out of delivery system 100 in an unpredictable manner that would impact deployment accuracy. Since unintended movement, i.e., bunching up or elongation, is avoided, stent-graft prosthesis 150 doesn't change length and deploys where intended with increased accuracy.

The radial dimension of the plurality of protrusions 132 is sufficient to provide enough friction to hold stent-graft prosthesis 150. However, the radial dimension of the plurality of protrusions 132 is preferably configured to minimize packing density in order to avoid interference with retraction of outer shaft component 102 during deployment of stent-graft prosthesis 150. In an embodiment hereof in which delivery system 100 has a profile between 14 and 20 French, the plurality of protrusions 132 may have a diameter between 0.030″-0.060″ with a height above covering 124 of 0.015″-0.030″. In another embodiment hereof in which delivery system 100 has a profile between 6 and 10 French, the plurality of protrusions 132 have a diameter between 0.010″-0.030″ with a height above covering 124 of 0.005″-0.015″.

Covering 124 may be formed by a suitable manufacturing method that intimately disposes covering 124 over distal segment 121 of inner shaft component 115 to be permanently and non-removably attached to inner shaft component 115 as described above. In an embodiment, covering 124 is formed via an overmolding manufacturing method in which inner shaft component 115 is positioned within a mold and covering 124 (including the plurality of protrusions 132) is overmolded by injection molding. In another embodiment, covering 124 is formed via reflow technology in which an extruded tubing segment formed from a material such as but not limited to PEBAX is positioned over distal segment 121 of inner shaft component 115, heat shrink tubing is positioned over the extruded tubing segment and then heat is applied to the heat shrink tubing in order to melt or reflow the extruded tubing segment over distal segment 121 of inner shaft component 115, thereby forming covering 124 from the reflowed extruded tubing segment. A dip method of manufacture may also be utilized to form covering 124.

In an embodiment hereof, covering 124 including the plurality of protrusions 132 may be equal or greater than the length of stent-graft prosthesis 150. Stated another way, in an embodiment hereof, a length of raised outer surface 130 of covering 124 is equal to or greater than a length of stent-graft prosthesis 150 and the length of stent-graft prosthesis 150 is disposed over raised outer surface 130. In another embodiment hereof, covering 124 including the plurality of protrusions 132 may be less than the length of stent-graft prosthesis 150. Stated another way, in another embodiment hereof, a length of raised outer surface 130 of covering 124 is less than a length of stent-graft prosthesis 150 and at least a distal portion of stent-graft prosthesis 150 is disposed over raised outer surface 130. For example, covering 124 including the plurality of protrusions 132 may extend only the length of a distal portion of stent-graft prosthesis 150. Further, different areas of the inner shaft component may be selectively built up with a covering according to embodiments hereof. For example, in an embodiment (not shown) in which the delivery system is for a bifurcated stent-graft having a long leg and a short leg, an area of the inner shaft component that corresponds to where the long leg of the bifurcated stent-graft extends past the short leg may be selectively built up with a covering such as covering 124 in order to push a portion of the long leg outward into contact with the outer shaft component.

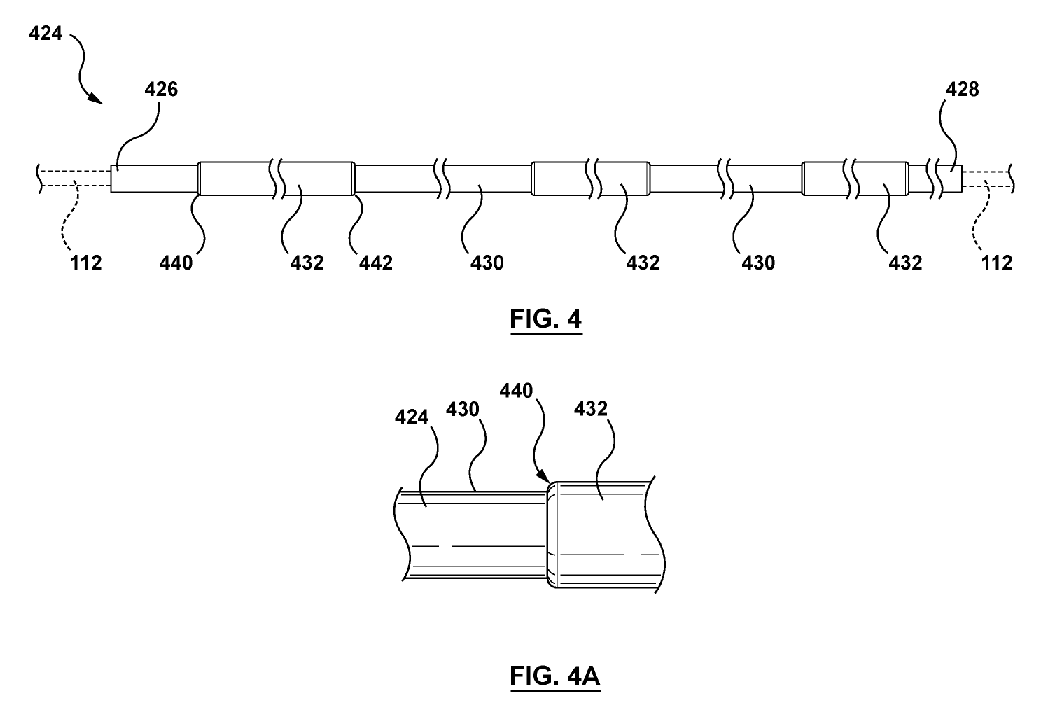

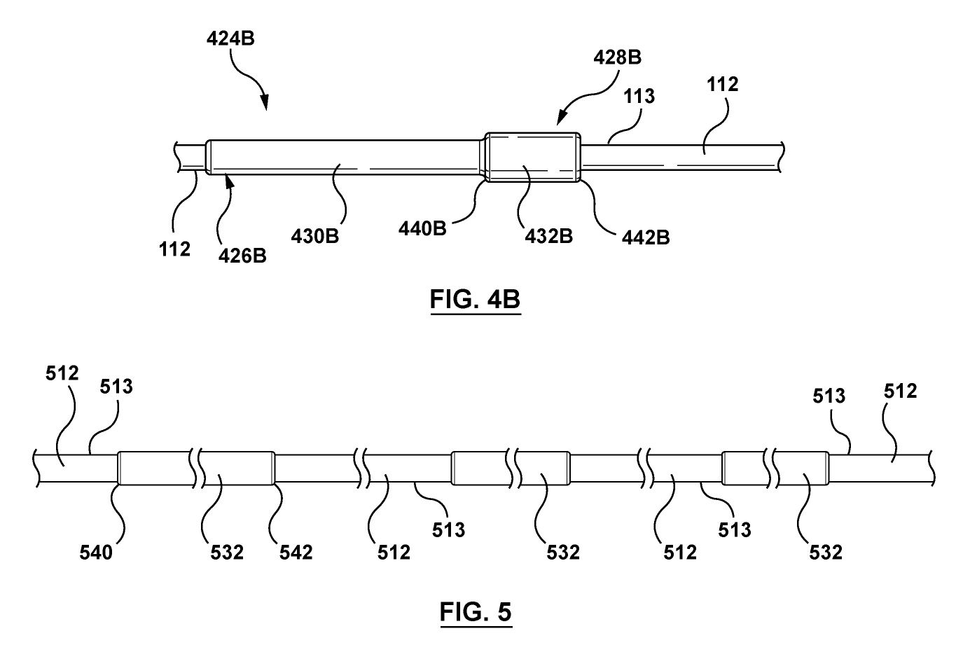

As described above, in an embodiment hereof, each protrusion 132 is a bump having a generally cylindrical, semi-spherical, dome-shaped, or conical configuration. However, protrusions 132 may have alternative configurations to provide sufficient friction to covering 124 to secure a longitudinal position of stent-graft prosthesis 150 relative to inner shaft component 112. For example, FIGS. 4 and 4A illustrate another embodiment hereof in which each protrusion of a plurality of protrusions 432 has a circumferential or cylindrical configuration. More particularly, each protrusion 432 is a circumferential band or ring that extends around a raised outer surface 430 of a covering 424. Covering 424 has a first or proximal end 426 and a second or distal end 428, while inner shaft component 112 is shown in phantom in FIG. 4. As best shown on FIG. 4A, each circumferential band includes opposing first and second circumferential edges 440, 442 that gradually taper to raised outer surface 430 of covering 424. Similar to protrusions 132, the plurality of protrusions 432 may be regularly or equally spaced over a length of covering 424.

In another embodiment hereof, only a single protrusion may be utilized to secure a longitudinal position of stent-graft prosthesis 150 relative to inner shaft component 112. For example, FIG. 4B illustrate another embodiment hereof in which a single protrusion 432B is disposed at a distal or second end 428B of a covering 424B. Protrusion 432B has a circumferential or cylindrical configuration. More particularly, protrusion 432B is a circumferential band or ring that extends around a raised outer surface 430B of covering 424B. Covering 424B is disposed over inner shaft component 112 and has a first or proximal end 426B and second or distal end 428B. Protrusion 432B has opposing first and second circumferential edges 440B, 442B. Since protrusion 432B is disposed at distal end 428B of covering 424B, first circumferential edge 440B gradually tapers to raised outer surface 430B of covering 424B while second circumferential edge 442B gradually tapers to an outer surface 113 of inner shaft component 112. In this embodiment, covering 424B may be considered a first, elongated raised circumferential or cylindrical segment disposed over the inner shaft component and protrusion 432B may be considered a second, relatively shorter circumferential or cylindrical segment disposed over the first, elongated raised segment.

In another embodiment hereof, the plurality of protrusions may be disposed directly onto the inner shaft component without a covering. More particularly, FIG. 5 illustrates an embodiment in which a series or plurality of protrusions 532 are disposed directly over an inner shaft component 512 such that protrusions 532 are separated by segments of exposed inner shaft component. Disposing protrusions 532 directly onto the inner shaft component without a covering minimizes the profile thereof, while the edges of protrusions 532 provide sufficient friction for retention of the stent-graft prosthesis disposed thereover. In the embodiment of FIG. 5, protrusions 532 have a circumferential or cylindrical configuration but other configurations may be utilized. More particularly, each protrusion 532 is a circumferential band or ring that extends around an outer surface 513 of inner shaft component 512. Each circumferential band includes opposing first and second circumferential edges. The plurality of protrusions 532 may be regularly or equally spaced over a portion of inner shaft component 512.

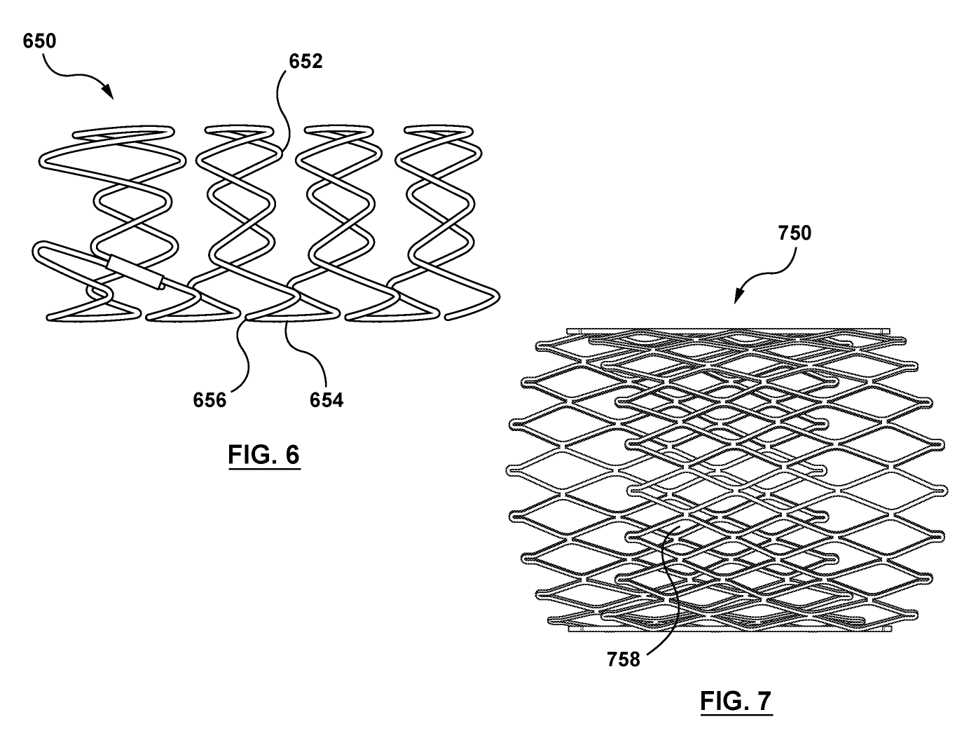

FIGS. 6 and 7 illustrate two exemplary self-expanding stent-graft prostheses that may be used with stent-graft delivery system 100. More particularly, FIG. 6 illustrates a radially-compressible stent or scaffold 650 that is operable to self-expand into apposition with an interior wall of a body vessel (not shown). As will be understood by one of ordinary skill in the art that stent 650 has a radially compressed configuration for delivery within a vasculature and a radially expanded configuration for deployment within a body lumen. FIG. 6 illustrates stent 650 in its radially expanded or deployed configuration. When in the radially compressed configuration, outer shaft component 102 of delivery system 100 is slidingly disposed over distal segment 121 of inner shaft component 112 for holding self-expanding stent-graft prosthesis 650 in the compressed delivery configuration. Although not shown in FIG. 6 for sake of clarity, it will be understood by one of ordinary skill in the art that stent 650 may be coupled to a tubular graft (not shown) formed from any suitable graft material, for example and not limited to, a low-porosity woven or knit polyester, DACRON material, expanded polytetrafluoroethylene, polyurethane, silicone, or other suitable materials. In another embodiment, the graft material could also be a natural material such as pericardium or another membranous tissue such as intestinal submucosa. Stent 650 may be coupled to the tubular graft by stitches or other means known to those of skill in the art. When stent 650 is used for treating an aneurysm, stent 650 has sufficient radial spring force and flexibility to conformingly engage stent 650 with the body lumen inner wall, to avoid excessive leakage, and prevent pressurization of the aneurysm, i.e., to provide a leak-resistant seal. Although some leakage of blood or other body fluid may occur into the aneurysm isolated by stent 650, an optimal seal will reduce the chances of aneurysm pressurization and resulting rupture.

Stent 650 is a helical stent and includes an elongated strut 652 coiled or spiraled into a series of windings that form a cylindrical profile. Strut 652 has a sinusoidal pattern defined by a plurality of crowns or bends 656 and a plurality of straight segments 654 with each crown 656 being formed between a pair of opposing straight segments 654. In an embodiment hereof, crowns 656 are not attached to each other such that stent 650 is very flexible and easy to compress axially. As such, coverings or loading pads described herein aid in preventing undesired axial compression or bunching up of stent 650 during loading and/or deployment thereof. In an embodiment hereof, stent 650 may have a helically-wound configuration as described in U.S. Pat. No. 8,998,975 to Rowe, hereby incorporated by reference in its entirety, or another helically-wound configuration as will be understood by one of ordinary skill in the art. In another embodiment, there may be connectors between at least one pair of crowns 656 in order to increase the stent column strength when compressed into the delivery system such as, for example, shown and described in U.S. Pat. No. 9,060,889 to Bliss et al. hereby incorporated by reference in its entirety. In another embodiment shown in FIG. 7, stent or scaffold 750 may be a unitary tubular component such as but not limited to a laser cut tubular stent defining openings 758. As will be understood by one of ordinary skill in the art, the stent or scaffold of a stent-graft prosthesis may have other configurations such as a series of sinusoidal patterned rings coupled to each other to form a self-expanding stent.

Although outer shaft component 102 is described herein as an elongate tube or sheath, it will be understood by one of ordinary skill in the art that outer shaft component 102 may have other configurations such as but not limited to configurations in which only a distal capsule portion thereof moves or is retracted during deployment of the stent-graft prosthesis. Thus, outer shaft component 102 may include a capsule component formed as a separate component from the elongate tube or sheath as described in U.S. Patent Publication No. 2011/0245917 to Savage et al., U.S. Patent Publication No. 2011/0251675 to Dwork, U.S. Patent Publication No. 2011/0251681 to Shipley et al., U.S. Patent Publication No. 2011/0251682 to Murray, III et al., and/or U.S. Patent Publication No. 2011/0264202 to Murray, III et al., each of which is herein incorporated by reference in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A catheter for delivering a self-expanding stent-graft prosthesis comprising:
an inner shaft component having a distal segment over which the stent-graft prosthesis is loaded in a compressed delivery configuration, wherein the distal segment has a covering that is intimately disposed thereover, the covering forming a raised outer surface on the distal segment and including at least one protrusion that extends radially outward from the raised outer surface; and
an outer shaft component slidingly disposed over the distal segment of the inner shaft component for holding the stent-graft prosthesis in the compressed delivery configuration,
wherein in the compressed delivery configuration the stent-graft prosthesis is disposed over and makes contact with the covering of the distal segment of the inner shaft component, such that at least the at least one protrusion provides sufficient friction to the covering to secure a longitudinal position of the stent-graft prosthesis relative to the inner shaft component.

2. The catheter of claim 1, wherein the at least one protrusion is a circumferential band that extends around the raised outer surface.

3. The catheter of claim 2, wherein the circumferential band includes opposing first and second edges that taper to the raised outer surface.

4. The catheter of claim 1, wherein the at least one protrusion includes a plurality of protrusions and each protrusion is a bump having a generally cylindrical, semi-spherical, dome-shaped, or conical configuration.

5. The catheter of claim 1, wherein the at least one protrusion includes a plurality of protrusions and the plurality of protrusions are regularly spaced over a length of the raised outer surface.

6. The catheter of claim 1, wherein the covering is an outer layer formed on the inner shaft component.

7. The catheter of claim 1, wherein the inner shaft component is formed from a tubing of a first material and the covering is formed from a second material different from the first material.

8. The catheter of claim 7, wherein the second material is elastomeric.

9. A stent-graft delivery system comprising:
an inner shaft component having a proximal segment and a distal segment, wherein the distal segment has a plurality of protrusions that radially extend away from an outer surface of the inner shaft component;
a self-expanding stent-graft prosthesis disposed over the distal segment, the self-expanding stent-graft prosthesis having a radially compressed configuration for delivery within a vasculature and a radially expanded configuration for deployment within a body lumen; and
an outer shaft component slidingly disposed over the distal segment of the inner shaft component for holding the self-expanding stent-graft prosthesis in the radially compressed configuration,
wherein the plurality of protrusions secure a longitudinal position of the self-expanding stent-graft prosthesis relative to the inner shaft component during loading and deployment of the self-expanding stent-graft prosthesis.

10. The stent-graft delivery system of claim 9, wherein each protrusion is a circumferential band that extends around the inner shaft component.

11. The stent-graft delivery system of claim 10, wherein the circumferential band includes opposing first and second edges that taper to the inner shaft component.

12. The stent-graft delivery system of claim 9, wherein the distal segment has a raised outer surface relative to the proximal segment and the plurality of protrusions radially extend away from the raised outer surface.

13. The stent-graft delivery system of claim 12, wherein each protrusion is a bump having a generally cylindrical, semi-spherical, dome-shaped, or conical configuration or each protrusion is a circumferential band that extends around the raised outer surface.

14. The stent-graft delivery system of claim 12, wherein an elastomeric layer attached to the distal segment forms the raised outer surface and the plurality of protrusions.

15. The stent-graft delivery system of claim 14, wherein the inner shaft component is formed from a tubing of a non-elastomeric material.

16. The stent-graft delivery system of claim 12, wherein a length of the raised outer surface is equal to or greater than a length of the self-expanding stent-graft prosthesis and the length of the self-expanding stent-graft prosthesis is disposed over the raised outer surface.

17. The stent-graft delivery system of claim 12, wherein a length of the raised outer surface is less than a length of the self-expanding stent-graft prosthesis and at least a distal portion of the self-expanding stent-graft prosthesis is disposed over the raised outer surface.

18. The stent-graft delivery system of claim 12, wherein the plurality of protrusions are regularly spaced over a length of the raised outer surface.

19. A method of loading a self-expanding stent-graft prosthesis into a delivery catheter, the method including the steps of:
radially compressing a self-expanding stent-graft prosthesis onto a loading pad on a distal segment of an inner shaft component of the delivery catheter, the loading pad being intimately disposed over the distal segment and defining a raised outer surface of the distal segment and a plurality of protrusions that extend radially away from the raised outer surface;
positioning the inner shaft component with the self-expanding stent-graft prosthesis radially compressed thereon within a lumen of an outer shaft component, wherein at least the plurality of protrusions secure a longitudinal position of the self-expanding stent-graft prosthesis relative to the inner shaft component such that the self-expanding stent-graft prosthesis does not move relative to the inner shaft component while the inner shaft component is being positioned within the lumen of the outer shaft component.

20. The method of claim 19, wherein the step of positioning the inner shaft component within the lumen of the outer shaft component includes pulling or pushing the inner shaft component into the lumen of the outer shaft component.
